(19) United States
(12) Patent Application Publication
(10) Pub. No.: US 2024/0303582 A1
(43) Pub. Date: Sep. 12, 2024

(54) SHIPMENT DATA MANAGEMENT SYSTEM FOR PHARMACEUTICAL COMPANIES

(71) Applicant: Navneet Kumar, Lexington, MA (US)

(72) Inventor: Navneet Kumar, Lexington, MA (US)

(73) Assignee: Myra EB Systems, Boxborough, MA (US)

(21) Appl. No.: 18/614,706

(22) Filed: Mar. 24, 2024

Related U.S. Application Data
(60) Provisional application No. 63/489,106, filed on Mar. 8, 2023.
(60) Provisional application No. 63/490,247, filed on Mar. 15, 2023.
(60) Provisional application No. 63/490,509, filed on Mar. 15, 2023.

(51) Int. Cl. G06Q 10/0832; G06Q 10/083; G06Q 10/087

(52) U.S. Cl. CPC G06Q 10/0832; G06Q 10/0838; G06Q 10/087

(57) ABSTRACT

The shipment platform offers an easy-to-use and streamlined method of documenting drug shipment tracking and management and ensuring a full trail of changes made to shipment requests. Here, you can input and view all shipment-related information, providing a centralized source of information and truth for team members tracking any drug shipments saving time on logistics-related communications. With the use of AI/ML systems, we can predict shipments that will be an issue in the future, potential delays, and costs associated with a shipment.

### FIELD OF INVENTION

The amount of data needed to be aggregated, organized, and analyzed in the pharmaceutical industries is massive and therefore, a specially designed IT system that handles Stability Data Management and a plurality of Shipping Information is needed to present the information to the users in a clear and simple way, to enable decisions to be made based on the available information. The industry is in need of an IT system that keeps data organized as well as create critical trend analyses quickly and provide them to the users making the decisions based on the available data. Lack of system results in non compliance and lost shipments worth millions of dollars.

### BACKGROUND INFORMATION

In the industry, the current systems need to be able to track shipments of pharmaceuticals to ensure that the pharmaceuticals do not expire before they are made available for patients. This is complicated by fact that different pharmaceuticals have different shelf lives and storage requirements. A particular pharmaceutical could have a shelf life of 6 months while another one being shipped could have a shelf life of 1 year, or a pharmaceutical could have a particular temperature requirement while another could have a particular humidity requirement. Therefore a system for tracking the various pharmaceuticals parameters combined with shipping information is needed in the industry.

### SUMMARY OF INVENTION

This application claims benefits from provisional patent applications No. 63/489,106, 63/490,247, and 63/490,509 and these applications are incorporated in their entirety into this application. The system is designed to manage the shipments of various drug substances, drug products, and samples in the pharmaceutical industry. The system is a very specialized and built with a focus on specific requirements of pharmaceutical products and materials.

The system is developed to enable users to generate shipment requests in a way that all the details associated with the shipments can be stored, retrieved, and referred from a single system. The shipment module allows users to add all the details relevant to a Drug Substance and Drug Product like Shipping company details, material type, material origination and destination, temperature conditions, etc. Users can fill in the details, attach associated documents, and send the shipment form along with all the details and documents to the Shipment Service provider as an email. Users can also save and download the shipment form as a PDF and manually send it to the shipment service provider. Details and documents associated with all the past shipments can be found on the ‘View Shipment’ Screen. User authentication is required to access the system, which means only registered users can log in using their valid credentials. Users from an organization can access the shipment forms created by the members of the same organization. The system supports two types of users, ones that can fill out and submit a shipping form and ones with view on access.

### BRIEF DESCRIPTIONS OF FIGURES

FIG. 1 shows an overview of the system.

FIG. 2 shows a flow chart of the system.

FIG. 3 shows a login interface.

FIG. 4 shows a Dashboard interface.

FIG. 5 shows a Templates interface.

FIG. 6 shows a Sites interface.

FIG. 7 shows a Shipment interface.

FIG. 8 shows an interface for inputting shipment data.

FIG. 9 shows an interface for inputting shipment data.

FIG. 10 shows a View Shipments interface.

FIG. 11 shows a View Shipment interface.

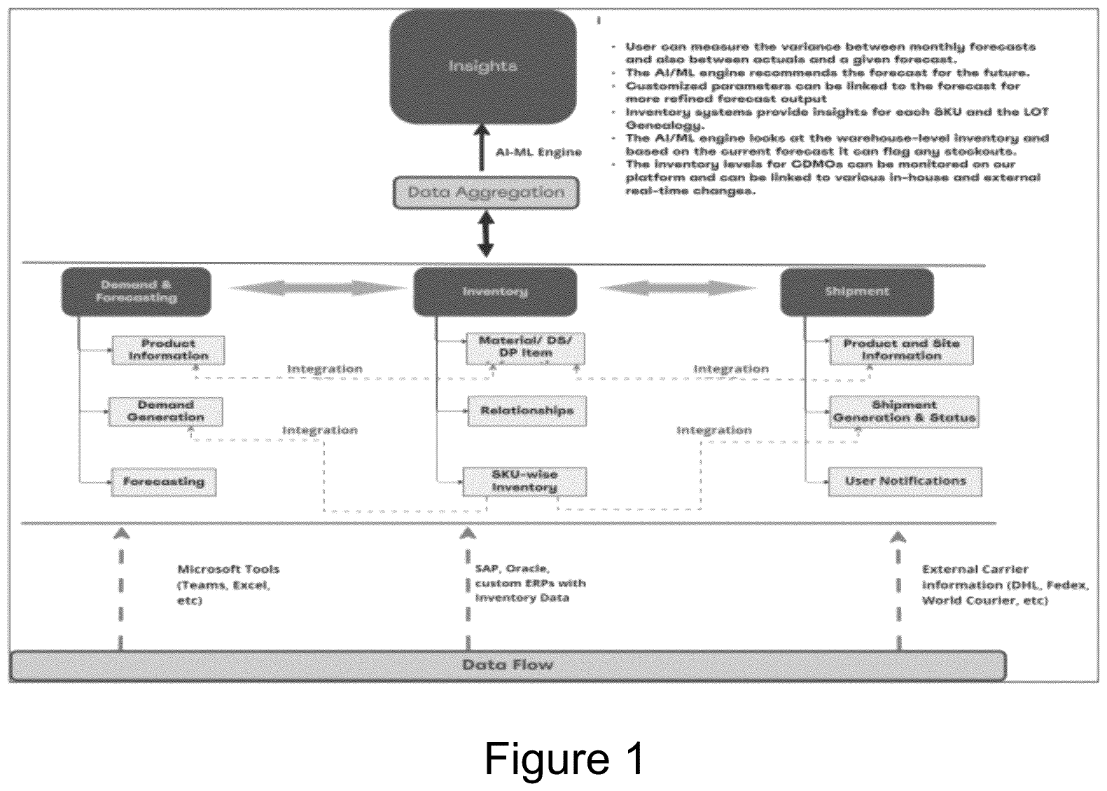

FIG. 1 shows an overview showing the different layers of the proposed system. At the lowest layer, the raw inventory data is inputted into the system by downloading it or having it uploaded from the warehouses, regions, countries, continents, or any other location that houses the pharmaceuticals. This data can take the form of information from Microsoft Products (Teams, Excel, etc.), SAP, Oracle, custom ERPs with inventory data, and External Carrier Information (DHL, FedEx, World Courier, etc.).

At the middle layer, the raw inventory data is aggregated and analyzed by different modules that can communicate both with each other and the layers above and below. The inventory module tracks raw materials, finished pharmaceuticals, locations, and SKU information for pharmaceuticals. This information is integrated into the Demand and Forecasting Module. The demand and forecasting module processes inventory information for wherever the pharmaceuticals are stored and generates forecasting information based on previous demand data, current demand data, and previously generated forecasting information. The generated forecast data is also integrated into the Inventory and Shipment modules. The Shipment Module tracks where raw materials and finished pharmaceuticals currency are located, where the items are currently scheduled to be delivered or currently in transit to. The shipment module can use and generate user notifications to inform users and the system as to locations and delivery points for all items currently in the shipping process.

The shipment systems collects and aggregates the load by product id (item no.), site, expiration, quantity, and batch. In real-time, shipment systems collect and aggregate information from various carriers such as FedEx, World Courier, Bio-care, or any courier able to ship pharmaceuticals. The shipping systems act as a data repository for COAs, SDS sheets, and other important templates required for shipments, and the shipment system aggregates data around notifications to understand better how fast the shipment has moved and where there were delays. The inventory system grabs data from various sources, such as warehouses and ERP systems. Compared to conventional ERP, the correlation between material, drug substance, and the finished product is very intuitive. The aggregation of LOT Geneology is the most appreciated feature that is not readily available in the current ERP system, which is needed in product recalls and product investigations. The shipment system provides the right level of access and notification to get insights on the real-time shipment status, which is done otherwise through heavy email exchanges and excel sheets. The AI/ML engine can classify shipments and sites that get delayed or take longer than expected. Integrating our shipment and inventory modules gives real-time updates to internal and external stakeholders using our platform. Our APIs allow external stakeholders to manage shipments and inventory to co-manage the information in real time, which significantly increases efficiency and allows optimized supply chain management.

At the highest level, the system employs Artificial Intellegence (AI) or Machine Learning (ML) to generate product stability information to be reintegrated into the middle layers. As pharmaceuticals must have a required shelf life to enable the providers at the end of the supply line to have a pharmaceutical that will last until needed by a patient. This shelf life is different for different pharmaceuticals, based on the compounds used, care needed (temperature, humidity, etc.), and rules and regulations for the given area where the pharmaceuticals are being distributed. This shelf life could be 6 months, 1 year, or any other amount of time that is required by the particular pharmaceutical. Using the AI or ML information generated by the Insight Module, a user can quickly have access to the information in an easy to read format. The information could be presented in graphs, visual representations, or text depending on the preference of the user. The user could receive alerts or notifications in the form of texts, emails, pages, calls, IMs, program pop up or overlays, banners, or any way of alerting a person to incoming information.

The Insight Module allows the user to measure and view the variance between monthly (or any time window) forecasts, including comparing past and current forecasts. The Insight Module uses the AI or ML system to generate a recommended forecast for the future, which could be any window of time selected by a user (days, weeks, months, years, etc.). The user can input customized parameters, which are then linked to the forecasting data to better refine the forecasts and the information presented to the user. The module also provides information about each SKU and LOT Genealogy for all the pharmaceuticals, which enables the system to track the stability data. The module can also inspect the quantities of pharmaceuticals being housed at every warehouse, and using the generated forecasts determine if any pharmaceuticals should be flagged as possibly running out of stock of and pharmaceutical. This module enables the system to create inventory levels for CDMOs for a pharmaceutical at any location in the supply chain and uses in-house and external realtime changes to predict the future stock levels at any point in the supply chain.

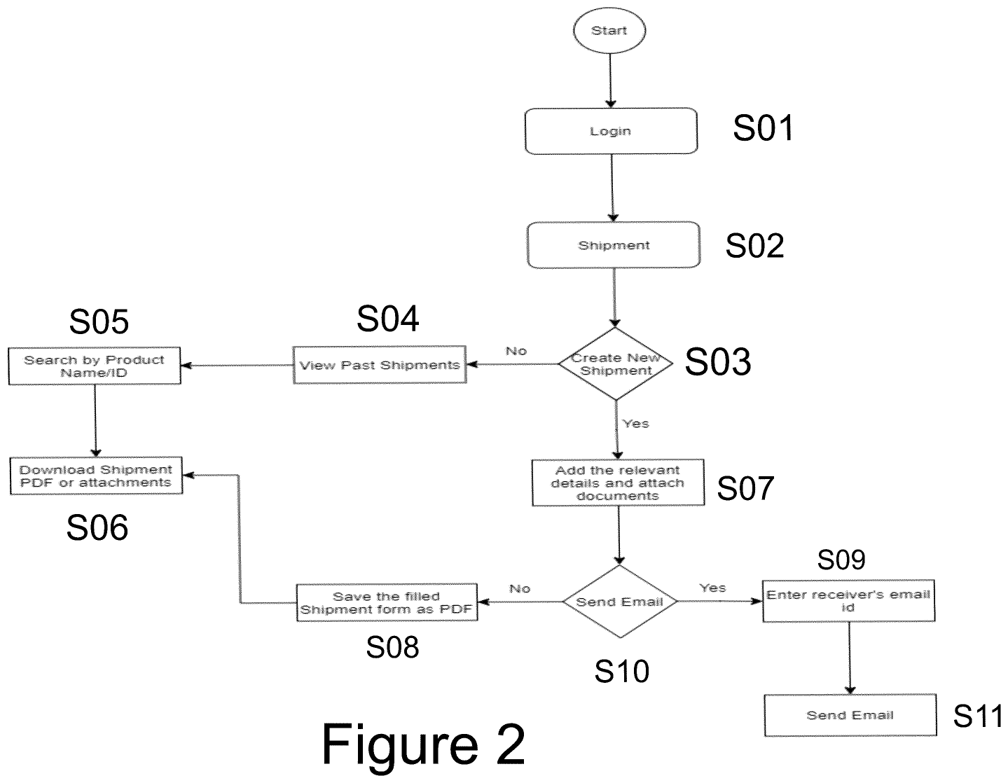

FIG. 2 shows a flow chart for the shipping system. At step 301, the user logs into the system, which allows them to access the shipping system at step S02. The user can choose to create a new shipments or view past shipments at step S03. In steps S04 and S05, the user can search through past shipments by using search term, and then the user can download any stored information for that shipment. If the user opts to created a new shipment, they are presented with an interface that allows them to input the shipping details and add any attachments at step S07. At step S10, the user can choose to get an email conformation, but any type of conformation would work including, but not limited by, texts, saving the information as a PDF, IMs, and pages.

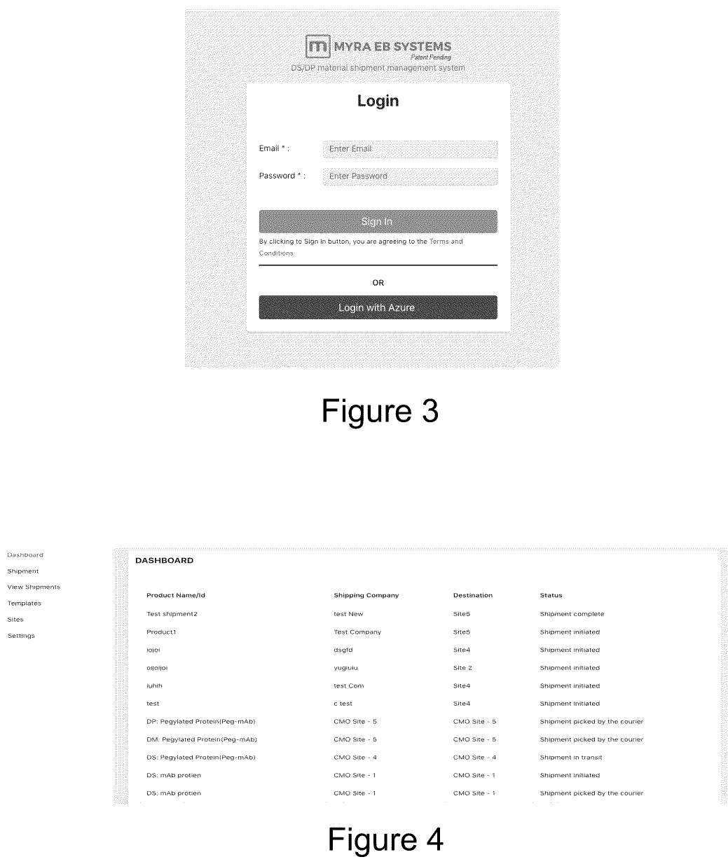

FIG. 3 shows a login interface that the user can input their email address and password. The login could be an email address, user ID, name, or code, while the password could be biometric like a fingerprint, iris, or face scan, voice imprint, password, pin number, or any other identifying mode.

FIG. 4 shows the interface enabling the user to view the dashboard, which provides information to track via the system, which allows the user to input the information shown to track the product in the supply chain. While the screen shot is shown as a preferred example of the system, variations of the screen shown could be used depending on the preferences of the user. The orientation of the options on the side of the screen could be located at any point on the page, in a pull down menu, animated buttons or options, in any order, or any variation that would allow for the same information to be presented to the user. The options shown could be a default setting of the software, which can be customized by the user and saved for future use. This customization would allow for buttons, graphs, images, and any information presented to be added or deleted from this page to only show the information required by the user. The software could also store a plurality of profiles for each user so that a user's preferences could be called up by pressing a button on the page or during a login screen presented when the software is started, allowing for the current user to enter their login information and/or password, select a stored profile, create a new profile, or select the default UI to save time. The profile or preferences could be used for each selectable window, or the profile or preferences could be used for every selectable window available while using the software. The default display could display a line graph as shown, any type of graph or visual representation of the information requested could be used, such as bar graphs, pie charts, 3D graphical representations, raw data, or any other means for displaying information to a user. While this description is applied to FIG. 1, the description would be applicable to every window discussed in the application. The actions button can used be adding information needed for the inventory analysis via a plus sign icon, minus sign icon, pencil icon, or any selectable means for bringing up additional menus. The dashboard would also present Information will include the name, shipment company, destination as well as the status of each shipment.

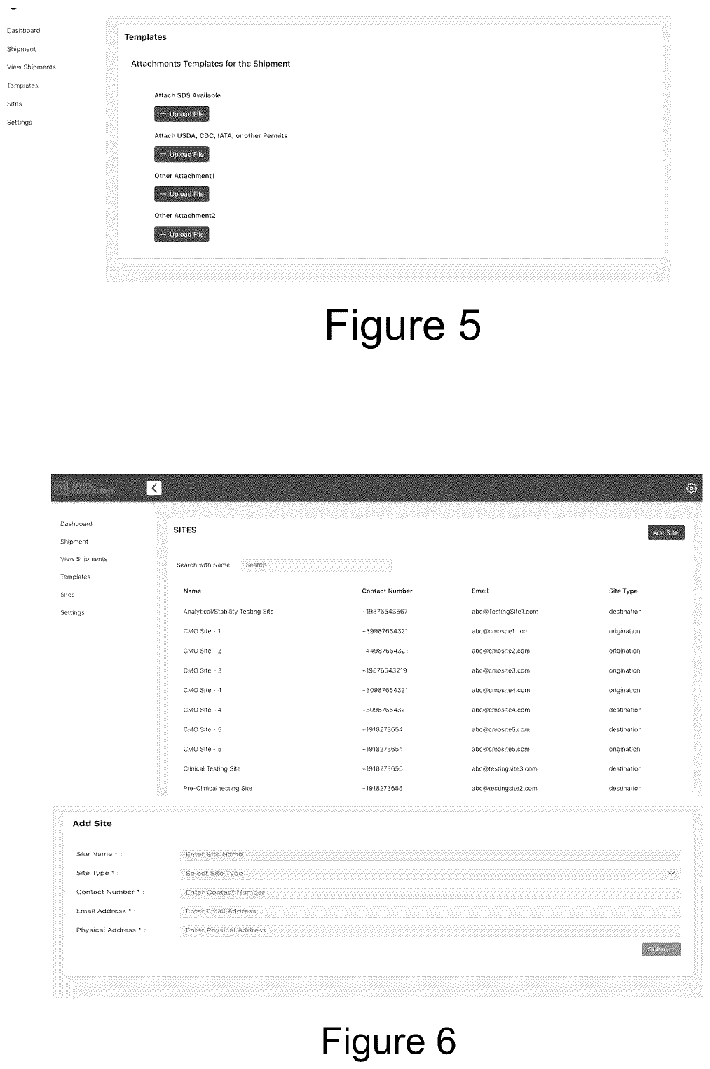

FIG. 5 shows the Templates option being selected from the dashboard. The templates section of the module is purely based on giving the user the best experience possible while using the system. To reach this section click on templates on the left-hand side of the screen. Here a user will be able to upload any company-specific forms onto the platform for future use. Once uploaded to the system, a link will be created to allow the user to download that form.

FIG. 6 shows the Sites option being selected from the dashboard. From the sites page, the first step in the process of inputting information into the system is the Sites tab. To reach this section click on Sites on the left-hand side of the screen. Here the user will find the page to document which sites are needed to function with the shipping system. To add a new site click on the purple add site button on the top right. After clicking on the add site button, the user will be prompted with a form to fill out. Fill in the information about the site you desire as well as the type of site. Whether it is a destination for the product you want to ship or an origin.

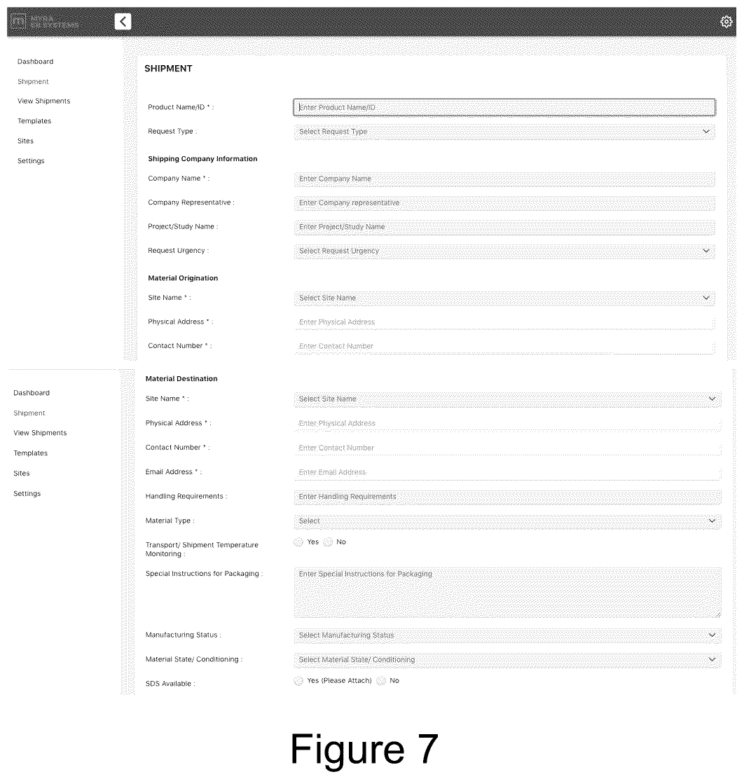

FIG. 7 shows the Shipment option being selected from the dashboard. To input, a shipment, click on the shipment tab on the left-hand side of the screen. The user will be taken to the shipment page where they must fill in the information requested by the form. The form requires the user to fill in all the necessary information to keep a diligent full record of the shipments made. The user follows the prompts and fill in the information as requested by the form(s).

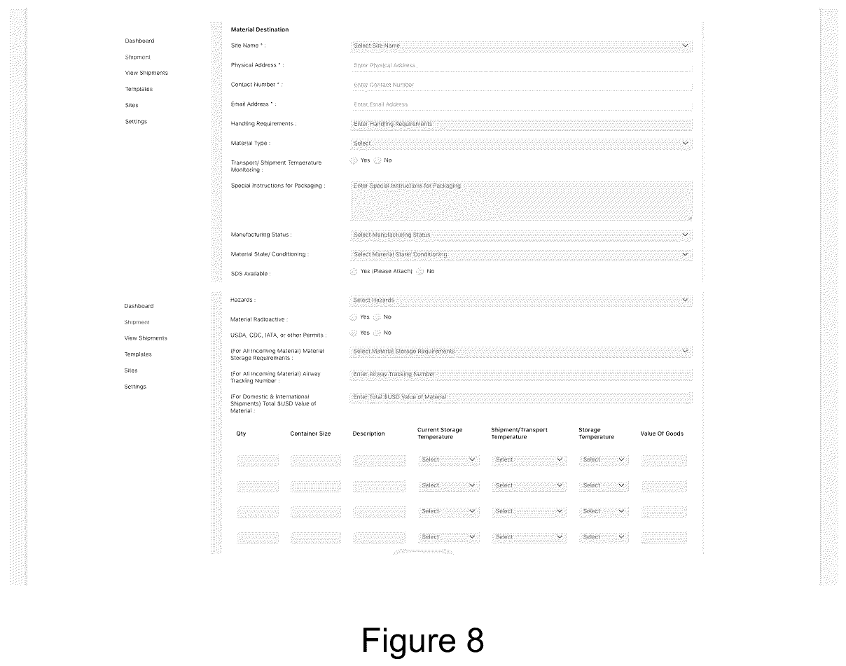

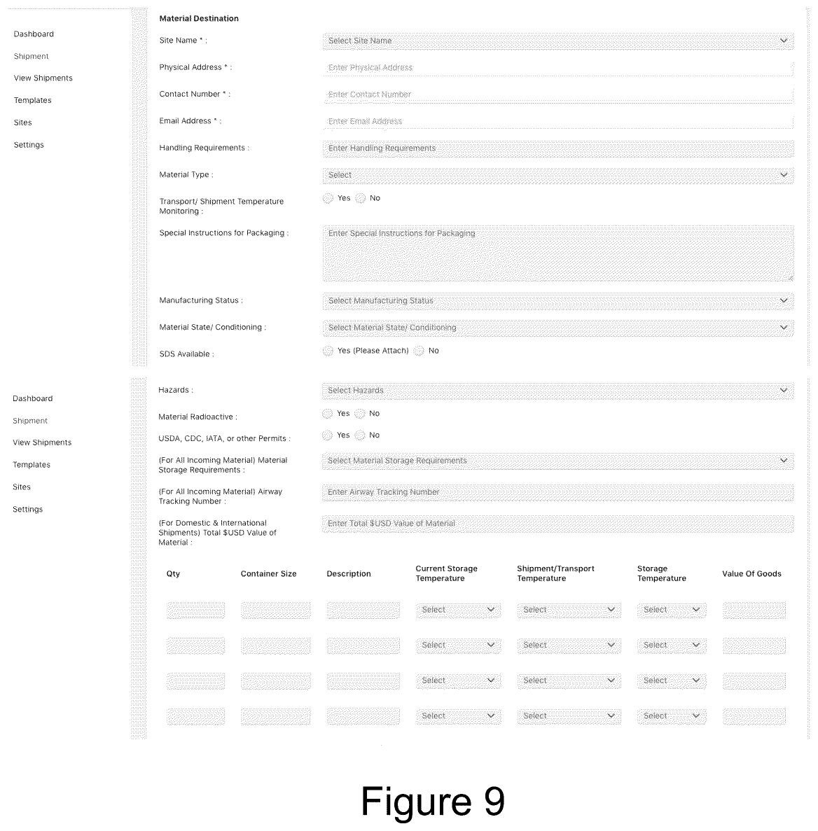

FIGS. 8 and 9 show interfaces for the user to input additional information for a shipment.

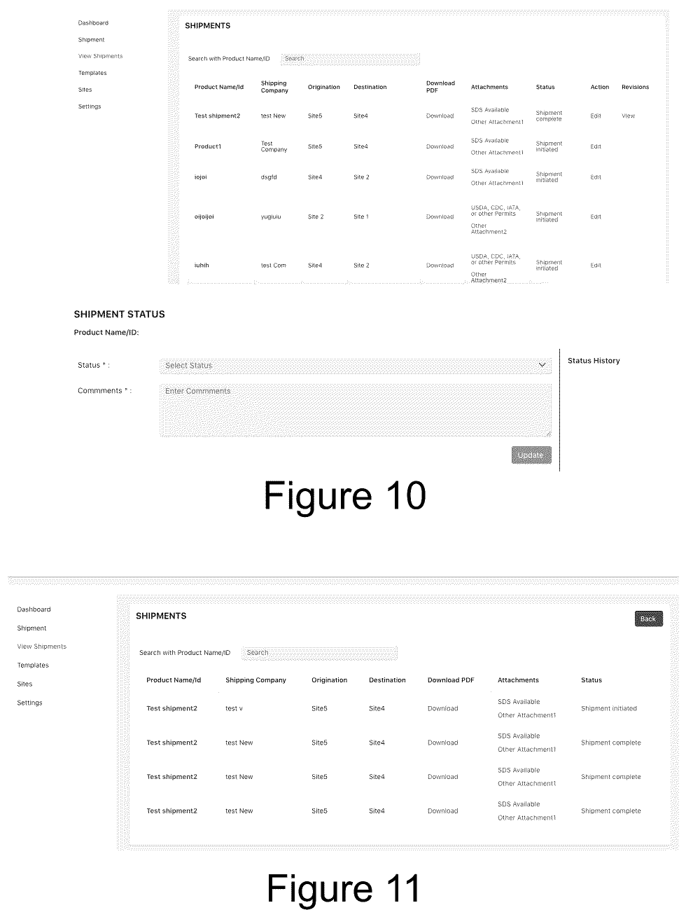

FIGS. 10 and 11 show the View Shipments option being selected from the dashboard. Here the user will be able to get a comprehensive view of the shipments that have been entered into the system. To view, click from the left-hand side of the screen the tab that says View Shipments. The user can perform several actions, such as clicking on the name of the product, the user will be brought to a view-only version of the form previously filled out to view the information, clicking on the download button the user will start a download of the PDF version of the corresponding shipment, clicking on any of the attachments connected, once again the user will start a download of the pdf version of said document, clicking on the link in the status section of the table, the user will be able to change the status of the payment bringing the user to, clicking on the edit button, the user will be brought to the form filled out for the corresponding shipment and can make edits to said form, and Finally, if there were any edits or changes to the shipment, under the revisions tab there is a view button. Once clicked the user will be brought to the history of changes made to the shipment

FIG. 21 shows the interfaces for manipulating the aggregated and generated data using the AI and ML tools. The tools is used to create insights into the stability for a particular pharmaceutical. The tools can use a system like ChatGPT-3 (or any conversational AI chat program) to ask real language questions for the system and output the information to the user in an easy to read and interpret the large amount of data aggregated and created using the system. For example the user could ask for the inventory of a drug, such as Embrel, at any location in the supply chain. Using that information the user could ask a follow up, such as whether the supply of Embrel at a particular location is in danger of running out, either from pharmaceuticals expiring, low supply, or issues up the supply chain. The user can use these queries to get common sense or real-world information from the system, which has the power to process more information that a human being could process on their own.

The devices mentioned above could be implemented using any type of processor architecture able to execute software including, but not limited to, x86, ENIAC, RISC, Pentium™, and Apple Silicon™. The software could be any type of code that is used to instruct a processor to perform instructions including, but not limited to, Python™, Java™, C+™, FORTRAN, and Assembly. The software could be stored on any type of non-transitory medium including, but not limited to, RAM, ROM, Flash Memory, SD cards, solid stated drives, spinning platter storage devices, Punch Cards, Piano Player Reels, Hard Drives, and physical servers.

We claim:

1. A system for determining pharmaceutical shipment information based on inventory and shipping times, comprising:
an interface for interpreting a user input for creating a shipping request for a particular pharmaceutical, wherein the system aggregates information stored in various databases and inputted pharmaceutical requirements and/or parameters to generate estimated shipping times, wherein:
the system analyzes the aggregated drug inventory locations and uses previous and current inventory data stored in a centralized database to compare the accuracies of the previous generated shipping times, wherein:
parameters inputted by the user comprising at least two of the previous inventory data, the previous forecast data, the current inventory data, pharmaceutical requirements, and the comparison data are used along with the current inventory to use AI or ML to generate a projected shipping information of a pharmaceutical to the user;
an interface to allow for the user to select different compounds, manufactured pharmaceuticals, and delivery methods and change parameters to determine how the project shipping information changes based on the parameter changes and display the change to the user.

2. A system for determining pharmaceutical shipment information based on inventory as claimed in claim 1, further comprising:
wherein the pharmaceutical parameters include the shelf life of a pharmaceutical.

3. A system for determining pharmaceutical shipment information based on inventory as claimed in claim 1, further comprising:
wherein data from shipping company databases are extracted to use in the shipping information generation.

4. A system for determining pharmaceutical shipment information based on real time data connection to databases as claimed in claim 1, further comprising:
wherein the data captured is at least one of Microsoft Products, SAP, Oracle, custom ERPs with inventory data, and External Carrier Information.

5. A system for determining pharmaceutical shipment information based on inventory as claimed in claim 1, further comprising:
wherein the changes are displayed via a visual representation.

6. A system for determining pharmaceutical shipment information based on inventory as claimed in claim 1, further comprising:
wherein the visual representation is a natural language text response.

7. A system for determining pharmaceutical shipment information based on inventory as claimed in claim 1, further comprising:
the information aggregated includes at least one of product id (item no.), site, expiration, quantity, and batch information.

8. A system for determining pharmaceutical shipment information based on inventory as claimed in claim 1, further comprising:
wherein the parameters for the pharmaceuticals stored at the database include at least one of: pharmaceutical expiration, shipping times, and pharmaceutical manufacturing time.
